(19) United States
(12) Patent Application Publication
(10) Pub. No.: US 2025/0050042 A1
(43) Pub. Date: Feb. 13, 2025

(54) DRY POWDER FORMULATIONS FILLED IN AN INHALER WITH IMPROVED RESISTANCE TO HUMIDITY

(71) Applicant: CHIESI FARMACEUTICI S.P.A., Parma (IT)

(72) Inventors: Barbara BASSI, PARMA (IT); Emanuele COSTA, PARMA (IT); Daniela COCCONI, PARMA (IT); Vincenzina SALVATO, PARMA (IT); Alan TWEEDIE, PARMA (IT); Marco DI CASTRI, PARMA (IT); Giuseppe Antonio MULAS, PARMA (IT); Sara BOTTINI, PARMA (IT)

(21) Appl. No.: 18/721,472

(22) PCT Filed: Dec. 19, 2022
(86) PCT No.: PCT/EP2022/086742

§ 371 Date: Jun. 18, 2024

(30) Foreign Application Priority Data
Dec. 21, 2021 (EP) 21216227.5

(51) Int. Cl. A61M 15/00; A61K 9/00; A61K 31/167; A61K 31/40; A61K 31/573; A61K 47/12; A61K 47/26

(52) U.S. Cl. CPC A61M 15/0091; A61K 9/0075; A61K 31/167; A61K 31/40; A61K 31/573; A61K 47/12; A61K 47/26; A61M 15/0026; A61M 2202/062; A61M 2202/064

(57) ABSTRACT

A drug product includes a multidose dry powder inhalation device. The device has a medicament chamber and a desiccant chamber adjacent to the medicament chamber, and a pharmaceutical composition therein. The pharmaceutical composition includes a pharmaceutically acceptable salt of formoterol, optionally in combination with a pharmaceutically acceptable salt of glycopyrronium and/or beclomethasone dipropionate. The desiccant chamber is filled with molecular sieves.

### FIELD OF THE INVENTION

The invention generally relates to inhalation drug products and methods of manufacturing the same.

### BACKGROUND OF THE INVENTION

Inhalers are hand-held portable devices that deliver medicaments directly to the lungs. One class of inhalers is passive dry powder inhalers (“DPI”). A passive DPI is a patient driven device wherein the action of breathing in through the device draws the powder formulation into the respiratory tract. DPIs are well recognized devices for the drug delivery to the lung for treatment of pulmonary and systemic diseases.

They can generally be divided in: i) single-dose (unit-dose) inhalers, for the administration of an individual dose of the active ingredient/s contained in capsule or blister loaded into the device and punctured by the patient immediately before use; ii) pre-metered multi-dose inhalers containing a series of blisters or capsules with the active ingredient/s formulation or iii) reservoir inhalers containing a larger amount of the powder formulation of active ingredient/s, corresponding to multiple doses sufficient for longer treatment cycles, which is metered from a storage unit just before inhalation. A formulation for DPI is commonly a powder blend of active ingredients and a bulk solid pharmacologically inert, physiologically acceptable diluent, such as lactose. The inhaled particle size of the active ingredients should be optimized to deliver the drug deep into the lung to achieve efficacy. This efficacious particle size typically lies between 1-6 micron whereas particles larger than this, i.e. 7-10 micron, tend to be deposited in the upper airways without reaching the site of action. It is well known that stability of the powder as well as the aerosol performances could be affected by environmental conditions, humidity in particular.

Therefore, it is desirable to control humidity within a DPI device.

In the art, usually silica gel has been used, see for example EP079066. However, its capacity is relatively low and it is not able to maintain the internal humidity stable (Lehto V P and Lankinen T, Int. J. Pharm. 275, 155, 2004).

A different desiccant was disclosed in WO 2008/040841 wherein the desiccant system comprises a salt such as magnesium chloride or potassium acetate.

Alternative approaches to control the moisture absorption by dry powder products have been shown in US 2008/0063719 and WO 2012/028662.

In the former one, an inhalable dry powder formulation of glycopyrrolate with a stability of at least 1 year under normal conditions was stored in packaging made from a material which itself has a moisture content less than 10%, preferably less than 5% and more preferably less than 3%, while in the latter one a device was disclosed comprising a hygroscopic material, and a package which encompasses the dry powder inhalation device and the hygroscopic material defining an enclosed volume therein, wherein the enclosed volume exhibits a Relative Humidity of from 20% to 40%.

Notwithstanding any potential progress that has been made regarding controlling humidity within the DPI device, there is still the need of more efficacious systems, in particular when active ingredients very sensitive to humidity are used, such as formoterol fumarate and glycopyrronium bromide.

Furthermore, there is the need of more efficacious systems to protect from humidity, advantageously when the drug-containing powder formulation is intended for being stored in sub-tropical and tropical countries.

### SUMMARY OF THE INVENTION

In a first aspect, the invention provides a drug product comprising a multidose dry powder inhalation device, in turn, comprising a medicament chamber and a desiccant chamber adjacent to the medicament chamber, said device having a pharmaceutical composition present therein, said pharmaceutical composition comprising a pharmaceutically acceptable salt of formoterol, optionally in combination with a pharmaceutically acceptable salt of glycopyrronium and/or beclometasone dipropionate (BDP), wherein the desiccant chamber is filled with molecular sieves.

In a preferred embodiment, the pharmaceutical composition comprises glycopyrronium bromide and/or beclometasone dipropionate (BDP) as further active ingredients.

In a particular embodiment, the multidose Dry Powder Inhaler comprises:
a casing (2) having a mouthpiece (4) and delimiting an inhalation channel connected to an opening (6) of the mouthpiece (4);
a container for storing a powdered medicament (medicament chamber) and placed in the casing (2);
a dispensing device placed in the casing (2) and configured to dispense unit doses of the powdered medicament from the container to the inhalation channel for inhalation through the mouthpiece (4);
a cover (3) engageable with the casing (2) to close the mouthpiece (4);
wherein the cover (3) comprises a sealing element (25) to further improve the resistance to humidity; and
whereby, when the cover (3) is engaged with the casing (2) and closes the mouthpiece (4), the main portion (26) of the sealing element (25) is coupled to the opening (6) to tight close said opening (6).

In another aspect, the invention provides a method for the treatment of a respiratory disorder. The method comprises administering to a patient by oral inhalation a pharmaceutically acceptable salt of formoterol, optionally in combination with a pharmaceutically acceptable salt of glycopyrronium and/or beclometasone dipropionate (BDP), using a drug product as described herein in the first aspect.

In a further aspect, the invention provides a process for manufacturing a drug product comprising a step of filling the medicament chamber of a multidose dry powder inhalation device with a pharmaceutical composition comprising a pharmaceutically acceptable salt of formoterol, optionally in combination with a pharmaceutically acceptable salt of glycopyrronium and/or beclometasone dipropionate (BDP), and the desiccant chamber of said device with molecular sieves.

The invention is also directed to a pharmaceutical composition in form of dry powder for inhalation comprising a fraction of fine excipient particles a) consisting of alpha-lactose monohydrate and magnesium stearate in amounts comprised between 98 and 99.0% and between 2.0 and 1.0% by weight, respectively; a fraction of coarse excipient particles b) consisting of alpha-lactose monohydrate having a particle size comprised between 210 and 360 micron and a tapped density comprised between 0.65 and 0.75 g/cm³, being the ratio between the fraction of fine excipient particles a) and the fraction of coarse excipient particles b) comprised between 2.5:97.5 and 7.5:92.5 by weight.

### Definitions

The term “pharmaceutically acceptable salt of glycopyrronium” refers to a salt of the compound (3S,2′R), (3R,2'S)-3-[(cyclopentylhydroxyphenylacetyl)oxy]-1,1-dimethylpyrrolidinium in approximately 1:1 racemic mixture.

The term “pharmaceutically acceptable salt of formoterol” refers to a salt of the compound 2′-hydroxy-5′-[(RS)-1-hydroxy-2 {[(RS)-p-methoxy-α-methylphenethyl] amino}ethyl]formanilide.

The term “beclometasone dipropionate” refers to the compound (8S,9R,10S,11S,13S,14S,16S,17R)-9-chloro-11-hydroxy-10,13,16-trimethyl-3-oxo-17-[2-(propionyloxy) acetyl]-6,7,8,9,10,11,12,13,14,15,16,17-dodecahydro-3H-cyclopenta[a]phenanthren-17-yl propionate.

The term “pharmaceutically acceptable salt” comprises inorganic and organic salts. Examples of organic salts may include formate, acetate, trifluoroacetate, propionate, butyrate, lactate, citrate, tartrate, malate, maleate, succinate, methanesulfonate, benzenesulfonate, xinafoate, pamoate, and benzoate. Examples of inorganic salts may include fluoride chloride, bromide, iodide, phosphate, nitrate and sulphate.

The “medicament chamber” is also defined in the art as “reservoir chamber” or “medicament container”.

The term “micronized” refers to a substance having a size of few microns.

The term “coarse” refers to a substance having a size of one or few hundred microns.

In general terms, the particle size of particles is quantified by measuring a characteristic equivalent sphere diameter, known as volume diameter, by laser diffraction.

The particle size can also be quantified by measuring the mass diameter by means of suitable known instrument such as, for instance, the sieve analyser.

The volume diameter (VD) is related to the mass diameter (MD) by the density of the particles (assuming a size independent density for the particles).

In the present application, the particle size of the active ingredients and of fraction of fine particles is expressed in terms of volume diameter, while that of the coarse particles is expressed in terms of mass diameter.

The particles have a normal (Gaussian) distribution which is defined in terms of the volume or mass median diameter (VMD or MMD) which corresponds to the volume or mass diameter of 50 percent by weight of the particles, and, optionally, in terms of volume or mass diameter of 10% and 90% of the particles, respectively.

Another common approach to define the particle size distribution is to cite three values: i) the median diameter d(0.5) which is the diameter where 50% of the distribution is above and 50% is below; ii) d(0.9), where 90% of the distribution is below this value; iii) d(0.1), where 10% of the distribution is below this value.

The span is the width of the distribution based on the 10%, 50% and 90% quantile and is calculated according to the formula.

$$Span = \frac{D [ v , 0.9 ] - D [ v , 0.1 ]}{D [ v , 0.5 ]}$$

In general terms, particles having the same or a similar VMD or MMD can have a different particle size distribution, and in particular a different width of the Gaussian distribution as represented by the d(0.1) and d(0.9) values.

Upon aerosolisation, the particle size is expressed as mass aerodynamic diameter (MAD), while the particle size distribution is expressed in terms of mass median aerodynamic diameter (MMAD) and Geometric Standard Deviation (GSD). The MAD indicates the capability of the particles of being transported suspended in an air stream. The MMAD corresponds to the mass aerodynamic diameter of 50 percent by weight of the particles.

The term “hard pellets” refers to spherical or semispherical units whose core is made of coarse excipient particles.

The term “spheronisation” refers to the process of rounding off of the particles which occurs during the treatment.

The term “good flowability” refers to a formulation that is easy handled during the manufacturing process and is able to ensure an accurate and reproducible delivering of the therapeutically effective dose.

Flow characteristics can be evaluated by different tests such as angle of repose, Carr's index, Hausner ratio or flow rate through an orifice.

In the context of the present application the flow properties were tested by measuring the flow rate through an orifice according to the method described in the European Pharmacopeia (Eur. Ph.) 7.3, 7^{th} Edition.

The expression “good homogeneity” refers to a powder wherein, upon mixing, the uniformity of distribution of a component, expressed as coefficient of variation (CV) also known as relative standard deviation (RSD), is less than 5.0%. It is usually determined according to known methods, for instance by taking samples from different parts of the powder and testing the component by HPLC or other equivalent analytical methods.

The expression “respirable fraction” refers to an index of the percentage of active particles which would reach the lungs in a patient.

The respirable fraction is evaluated using a suitable in vitro apparatus such as Andersen Cascade Impactor (ACI), Multi Stage Liquid Impinger (MLSI) or Next Generation Impactor (NGI), according to procedures reported in common Pharmacopoeias, in particular in the European Pharmacopeia (Eur. Ph.) 7.3, 7^{th} Edition.

It is calculated by the percentage ratio of the fine particle mass (formerly fine particle dose) to the delivered dose.

The delivered dose is calculated from the cumulative deposition in the apparatus, while the fine particle mass is calculated from the deposition of particles having a diameter <5.0 micron.

In the context of the present application, the formulation is defined as extrafine formulation when it is able of delivering a fraction of particles having a particle size equal or less than 2.0 micron equal to or higher than 20%, preferably equal to or higher than 25%, more preferably equal to or higher than 30% and/or it is able of delivering a fraction of particles having a particle size equal or less than 1.0 micron equal to or higher than 10%.

The expression “physically stable in the device before use” refers to a formulation wherein the active particles do not substantially segregate and/or detach from the surface of the carrier particles both during manufacturing of the dry powder and in the delivery device before use. The tendency to segregate can be evaluated according to Staniforth et al. J. Pharm. Pharmacol. 34,700-706, 1982 and it is considered acceptable if the distribution of the active ingredient in the powder formulation after the test, expressed as relative standard deviation (RSD), does not change significantly with respect to that of the formulation before the test.

The expression “chemically stable” refers to a formulation that, upon storage, meets the requirements of the EMEA Guideline CPMP/QWP/122/02 referring to ‘Stability Testing of Existing Active Substances and Related Finished Products’.

The term “surface coating” refers to the covering of the surface of the carrier particles by forming a film of magnesium stearate around said particles. The thickness of the film has been estimated by X-ray photoelectron spectroscopy (XPS) to be approximately of less than 10 nm. The percentage of surface coating indicates the extent by which magnesium stearate coats the surface of all the carrier particles.

The term “prevention” means an approach for reducing the risk of onset of a disease.

The term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. The term can also mean prolonging survival as compared to expected survival if not receiving treatment.

According to the Global Initiative for Asthma (GINA), “severe persistent asthma” is defined as a form characterized by daily symptoms, frequent exacerbations, frequent nocturnal asthma symptoms, limitation of physical activities, forced expiratory volume in one second (FEV₁) equal to or less than 60% predicted and with a variability higher than 30%.

According to the Global initiative for chronic Obstructive Lung Disease (GOLD) guidelines, “severe COPD” is a form characterized by a ratio between FEV₁ and the Forced Vital Capacity (FVC) lower than 0.7 and FEV₁ between 30% and 50% predicted. The very severe form is further characterized by chronic respiratory failure.

“Therapeutically effective dose” means the quantity of an active ingredient-administered at one time by inhalation upon actuation of the inhaler. Said dose may be delivered in one or more actuations, preferably one actuation (shot) of the inhaler.

“Actuation” refers to the release of an active ingredient from the device by a single activation (e.g. mechanical or breath).

To check stability, studies of pharmaceutical drugs shall be performed under different conditions according to the climatic conditions of the country. According to ICH guidelines for stability studies the world climate is divided in five different zones:

| Zone | Type of Climate |
|---|---|
| Zone I | Temperate zone |
| Zone II | Mediterranean/subtropical zone |
| Zone III | Hot dry zone |
| Zone IVa | Hot humid/tropical zone |
| Zone IVb | Hot/higher humidity |

As used in this specification and the appended claims, the singular forms “a”, “an”, “the” and “one” include plural referents unless the content clearly dictates otherwise. Thus, for example, reference to “a salt” includes two or more such salts; reference to “a constituent” includes two or more such constituents and the like.

### FIGURES

FIG. 1 shows a 3D view of a powder inhaler according to the present invention in an open configuration.

FIG. 2 shows an enlarged view of a portion of the powder inhaler of FIG. 1.

FIG. 3 is a plot showing the results obtained as described in Example 1: A) cumulative weight increment (mg, ordinates) over time (days, abscissae) due to moisture absorption in an inhaler comprising molecular sieves in the desiccant chamber (PROPAGROUP, grey line) compared to an inhaler comprising silica gel (STD, black line); B) a differential weight (mg, ordinates) over time (days, abscissae) due to moisture absorption in an inhaler comprising molecular sieves in the desiccant chamber (dotted bar) compared to an inhaler comprising silica gel (slash lines bar). In B) the higher is the bar, the greater is the reduction of moisture absorption.

FIG. 4 is a chart showing a differential weight over time due to moisture absorption of inhalers incorporating the separate different embodiments of the invention compared to a standard inhaler according to WO 2016/000983. The higher is the bar, the greater is the reduction of moisture absorption.

FIG. 5 shows a 3D view of an internal element of the device according to WO 2004/012801 or WO 2016/000983, the metering member, or shuttle, of the inhaler shaped like a plate.

FIG. 6 shows a view of the elastomer island embodiment applied at a metering member of FIG. 5.

FIG. 7A is an exploded 3D view of some internal components of the inhaler according to WO 2004/012801 or WO 2016/000983 including the gasket embodiment.

FIG. 7B shows a 3D view of one element of the powder inhaler from the view of FIG. 7A related to the gasket embodiment.

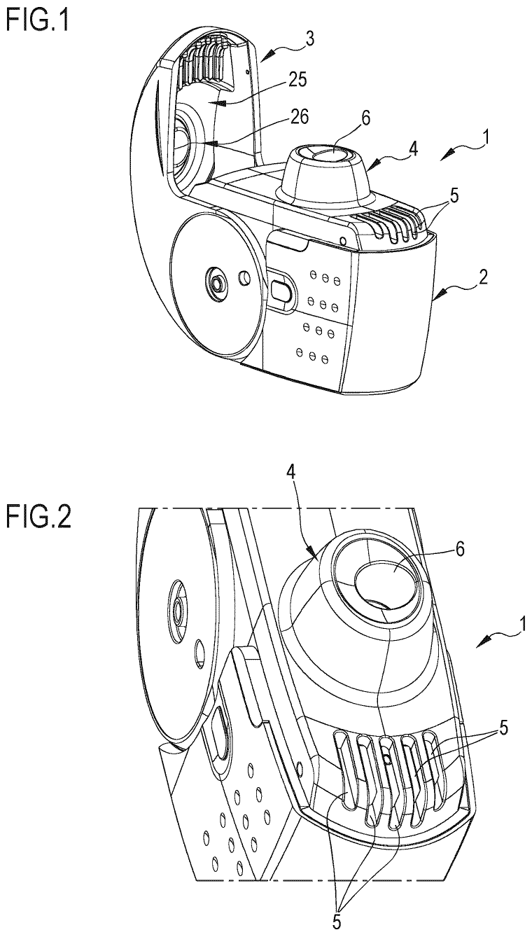

### DETAILED DESCRIPTION OF THE INVENTION

The invention will now be described with respect to the embodiments presented herein. Before describing the present invention in detail, it is to be understood that this invention is not limited to particularly exemplified structures, apparatus, systems, materials or methods, which as such may of course vary. Thus, although a number of apparatus, systems and methods similar or equivalent to those described herein can be used in the practice of the present invention, the preferred apparatus, systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

In a first aspect, the invention provides a drug product comprising a multidose dry powder inhalation device comprising a medicament chamber and a desiccant chamber adjacent to the medicament chamber, said device having a pharmaceutical composition present therein, said pharmaceutical composition comprising, as active ingredients, a pharmaceutically acceptable salt of formoterol, optionally in combination with a pharmaceutically acceptable salt of glycopyrronium and/or beclometasone dipropionate (BDP), wherein the desiccant chamber is filled with molecular sieves.

Said molecular sieves are made of a material with pores of uniform size and they can absorb small molecules such as water. Typically, they are made of alkaline salts of aluminosilicates, called zeolites, with pores having a diameter comprised from 2 to 50 angstrom, preferably from 3 to 20 angstrom. In a preferred embodiment, the diameter is 10 angstrom. Other suitable materials know in the art, such as aluminophosphates, porous glass or active carbon could advantageously be used. Artificial zeolites could also be used.

In a particular embodiment, the molecular sieves are contained in a Tyvek® bag able of being inserted in the desiccant chamber. Typically, the bag is inserted inside the desiccant chamber and after that the foil is sealed to seal the desiccant chamber itself. In an alternative embodiment, the desiccant chamber is filled with the molecular sieves in form of a single tablet.

The amount of molecular sieves will depend on the geometry and the volume of the desiccant chamber. In one embodiment, the amount of molecular sieve per bag is 0.2-1.5 g.

Advantageously, the desiccant chamber and the medicament chamber are separated by a permeable membrane.

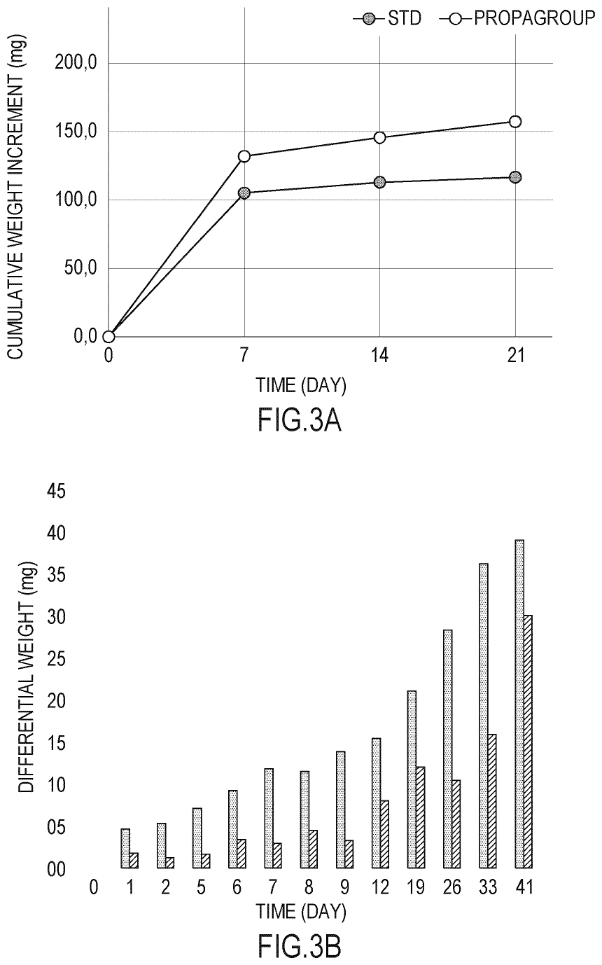

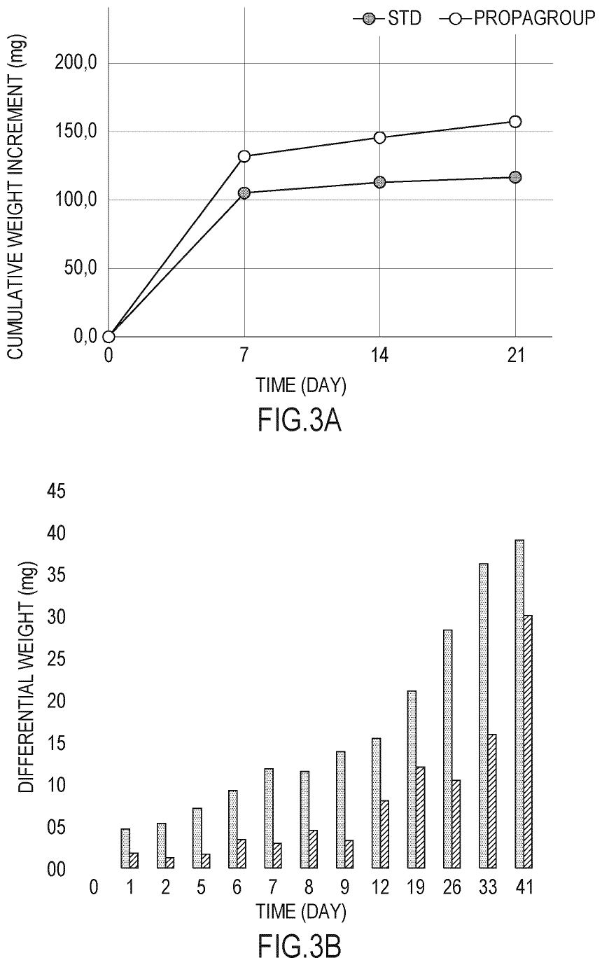

It has been found that molecular sieves are very efficient in increasing the speed of humidity extraction during the use of the inhaler. In fact, as reported in FIG. 3, molecular sieves absorb humidity much quicker than silica gel, allowing for a more dry environment of the desiccant chamber to be maintained.

Accordingly, the drug product is capable of exhibiting improved shelf life and more stable fine particle fraction as a result of the system of the invention.

In particular, the formulation filled in the device turned to be physically and chemically stable before use and during storage, while maintaining good homogeneity and flowability and a good respirable fraction over time.

In addition to the medicament chamber and the desiccant chamber, the Dry Powder Inhaler (DPI) shall comprise a mouthpiece through which the user could inhale the powder medicament.

Advantageously, it further comprises a case with a lower shell and an integral cover, being pivotably or rotatably coupled to the lower shell.

The cover could be opened to reveal the mouthpiece.

In a preferred embodiment, the dry powder inhaler comprises:
a casing (2) having a mouthpiece (4) and delimiting an inhalation channel connected to an opening (6) of the mouthpiece (4);
a container for storing a powdered medicament and placed in the casing (2);
a dispensing device placed in the casing (2) and configured to dispense unit doses of the powdered medicament from the container to the inhalation channel for inhalation through the mouthpiece (4);
a cover (3) engageable with the casing (2) to close the mouthpiece (4);
wherein the cover (3) comprises a sealing element (25) to further improve the resistance to humidity; and
whereby, when the cover (3) is engaged with the casing (2) and closes the mouthpiece (4), the main portion (26) of the sealing element (25) is coupled to the opening (6) to tight close said opening (6).

The sealing element (25) is made of a soft or a medium soft material and is more deformable and flexible than the material of the mouthpiece.

Advantageously, the sealing element (25) has a hardness between 10 Shore A and 60 Shore A, preferably between 20 Shore A and 50 Shore A, more preferably between 25 Shore A and 35 Shore A, more preferably of 30 Shore A.

The Shore A Hardness Scale measures the hardness of flexible mold rubbers that range in hardness from very soft and flexible, to medium and somewhat flexible, to hard with almost no flexibility at all. In some embodiments, the sealing element (25) is made of silicone, e.g. PlatSil® FS-20 mixed with PlatSil® GEL.

Alternative materials such as thermoplastic elastomers (TPEs) could be used. Suitable TPE may be selected from those of medical-pharmaceutical grade belonging to classes of styrene block copolymers, thermoplastic polyolefin elastomers, and thermoplastic polyurethanes. Preferably silicone is used.

Said element could have a thickness variable between about 0.2 and 5 mm, preferably 1 and 2 mm, and a shape that mimic the external geometry of the mouthpiece, in order to fill the gaps of the mouthpiece itself and avoid that humidity could enter through those gaps.

Typically, the sealing element is over-molded to the cover (3) or press-fitted in the cover (3) and optionally glued to the cover (3) or mechanically connected to the cover (3).

Advantageously, the casing (2) has at least one air inlet (5) in fluid communication with the inhalation channel to allow air intake at least when the user draws from the mouthpiece (4); wherein, when the cover (3) is engaged with the casing (2) and closes the mouthpiece (4), the main portion (26) of the sealing element (25) is coupled to the at least one air inlet (5) to tight close said at least one air inlet (5).

Exemplary Dry Powder Inhalers (DPIs) with a cover engageable with the casing to close the mouthpiece suitable to host the sealing element and with a desiccant chamber suitable for being filled with the molecular sieves are reported in WO 2004/012801 and WO 2016/000983 to which the reader is referred to for the drawings and numbering of the various parts, except for the numbering herein reported.

Advantageously, the cup of the medicament chamber of the DPI according to WO 2004/012801 or WO 2016/000983 is configured to deliver 180 doses rather than 120 doses, as it has been observed that the larger the cup, more mandatory is the request of having a more protective effect against the access of moisture.

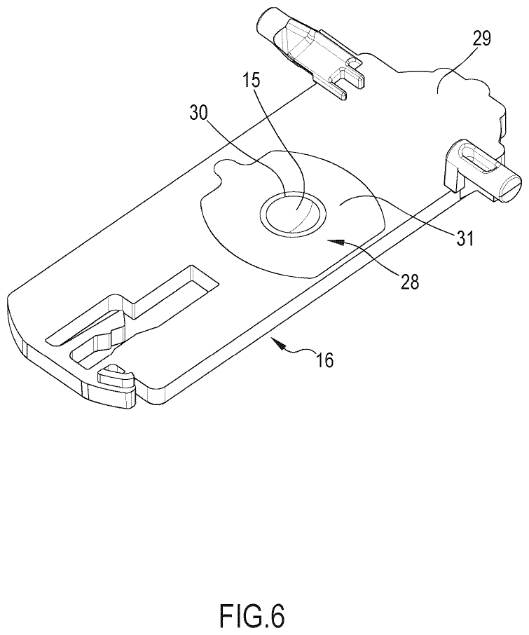

Typically, the dispensing device of the DPI according to WO 2004/012801 or WO 2016/000983 comprises a shuttle (FIG. 5; (16) of FIG. 6) having a dosing recess, wherein said shuttle is movable between a filling position, in which the dosing recess is in alignment with an opening of the container and faces said opening so as to be filled with a dose of the powdered medicament, and an inhalation position, in which the dosing recess is in alignment with the inhalation channel for enabling inhalation of the dose of the powdered medicament contained in the dosing recess through the mouthpiece.

As disclosed in a detailed manner in the co-pending application n. EP 21216553.4, in order to further reduce the access of humidity, the DPI further comprises a sealing device operationally active at a coupling zone of the shuttle with the container when the shuttle is in the filling position, said coupling zone circumscribing the dosing recess and the opening.

In a more preferred embodiment (elastomer island, FIG. 6), the sealing device comprises a deformable portion (31) surrounding the dosing recess (15) and connecting the dosing part (30) to the main part (29), wherein the deformable portion is lesser stiff than the main part (29) along a direction perpendicular to a lying plane of the shuttle; wherein at least one spring is interposed between the casing (2) and the dosing part and is configured to push said dosing part against the opening of the container by deforming the deformable portion of the shuttle when the shuttle is in the filling position.

According to said more preferred embodiment, the shuttle could be shaped like a plate and the main part (29) could have a first wall thickness (t1), wherein the deformable portion has a second wall thickness (t2) smaller than the first wall thickness (t1), optionally wherein a ratio t2/t1 of the second wall thickness (t2) to the first wall thickness (t1) is smaller than 0.5, optionally smaller than 0.3.

Advantageously, the dosing part could comprise a peripheral stiffening rib surrounded by the deformable portion, wherein the deformable portion has an average width (wₐᵥ) and a ratio t2/wₐᵥ of the second wall thickness (t2) to the average width (wₐᵥ) is smaller than 0.3, optionally smaller than 0.2, optionally smaller than 0.1.

Typically, the main part (29), the deformable portion (31) and the dosing part (30) are made in a single piece, optionally of plastic, optionally of acrylonitrile butadiene styrene (ABS). Preferably, the deformable portion is made of or comprises an elastomeric material, optionally a medical grade butyl rubber.

The dosing part (30) could comprise at least one stiffening rib on a side opposite the dosing recess.

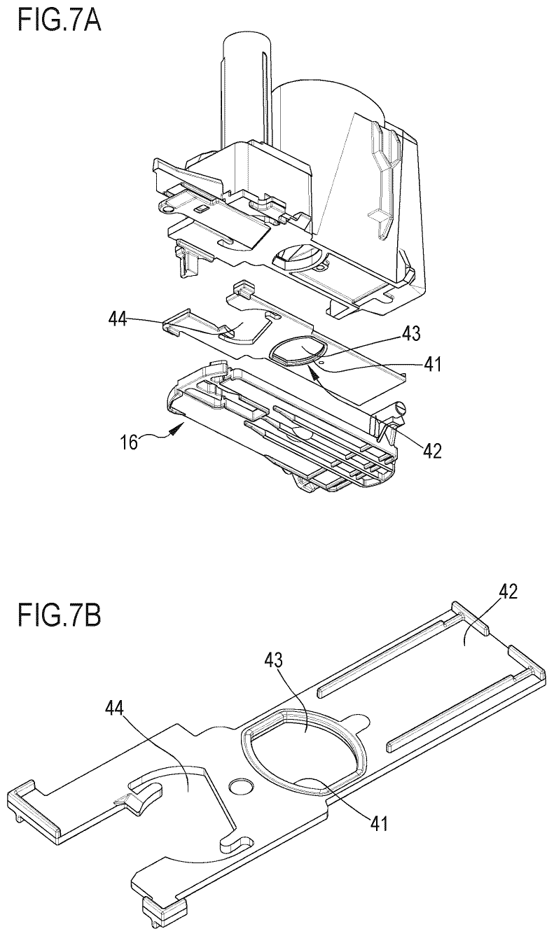

In an alternative embodiment (gasket, FIG. 7), the sealing device could comprise a gasket (41) placed at the coupling zone, at least when the shuttle is in the filling position.

In this embodiment, the Dry Powder Inhaler comprises a support plate (42) made of plastic, e.g. acetal resin. The support plate (42) is sandwiched between the container and the shuttle. The support plate (42) is anchored to the casing (2).

The support plate (42) has a through opening (43) and a through inhalation passage (44).

Advantageously, the gasket could be made of an elastomeric material, preferably selected from thermoplastic elastomers (TPEs) of medical-pharmaceutical grade or silicone, and the support plate is made of plastic, optionally acetal resin. Preferably, said gasket has a raised bead protruding towards the container and/or a raised bead protruding towards the shuttle.

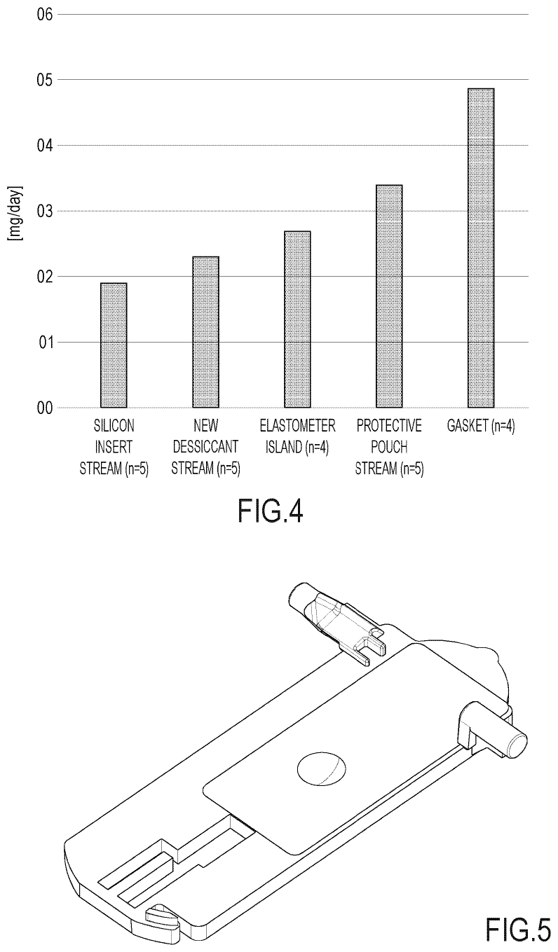

As it can be appreciated for FIG. 4, which shows a differential weight over time due to moisture absorption of inhalers incorporating separate different embodiments of the invention compared to a standard inhaler, both the elastomer island and the gasket are very effective in decreasing the absorption of moisture.

The pharmaceutical composition filling the medicament chamber of the multidose inhaler (DPI) shall be in form of dry powder. Advantageously, it comprises a fraction of fine excipients particles a), a fraction of coarse excipient particles b), and micronized particles of a pharmaceutically acceptable salt of formoterol, optionally in combination with a pharmaceutically acceptable salt of glycopyrronium and/or beclometasone dipropionate (BDP).

The fractions a) and b) are the “carrier” particles.

Preferably, the salt of formoterol is formoterol fumarate dihydrate, and the salt of glycopyrronium is glycopyrronium bromide.

In a preferred embodiment, the pharmaceutical composition comprises beclometasone dipropionate (BDP) and, even more preferably, glycopyrronium bromide as further active ingredients.

Advantageously, the fine and coarse excipient particles may consist of any pharmacologically inert, physiologically acceptable material or combination thereof; preferred excipients are those made of crystalline sugars, in particular lactose; the most preferred are those made of alpha-lactose monohydrate.

Preferably, the coarse excipient particles and the fine excipient particles are made of the same material and both consist of alpha-lactose monohydrate.

The coarse excipient particles of the fraction b) must have mass median diameter equal to or higher than 100 micron preferably equal to or greater than 125 micron, more preferably equal to or greater than 150 micron, even more preferably equal to or greater than 175 micron.

Advantageously, all the coarse particles have a mass diameter in the range 50-1000 micron, preferably comprised between 60 and 500 micron.

In certain embodiments of the invention, the mass diameter of said coarse particles might be comprised between 80 and 200 micron, preferably between 90 and 150 micron, while in another embodiment, the mass diameter might be comprised between 200 and 400 micron, preferably between 210 and 380 micron.

In a preferred embodiment of the invention, the mass diameter of the coarse particles is comprised between 210 and 360 micron.

In general, the person skilled in the art shall select the most appropriate size of the coarse excipient particles by sieving, using a proper classifier.

When the mass diameter of the coarse particles is comprised between 200 and 400 micron, the coarse excipient particles preferably have a relatively highly fissured surface, that is, on which there are clefts and valleys and other recessed regions, referred to herein collectively as fissures. The “relatively highly fissured” coarse particles can be defined in terms of fissure index or rugosity coefficient as described in WO 01/78695 and WO 01/78693, whose teachings are incorporated herein by reference, and they could be characterized according to the description therein reported. Advantageously, the fissure index of said coarse particles is of at least 1.25, preferably of at least 1.5, more preferably of at least 2.0, while the rugosity coefficient is of at least 1.01, preferably comprised between 1.02 and 1.3.

Said coarse particles may also be characterized in terms of tapped density or total intrusion volume measured as reported in WO 01/78695.

The tapped density of said coarse particles could advantageously be less than 0.8 g/cm³, preferably between 0.8 and 0.5 g/cm³. The total intrusion volume could be of at least 0.8 cm³, preferably at least 0.9 cm³.

Another way to express their density is to determine their dynamic density. It can be determined as reported in Example 3. The dynamic density for the aforementioned particles is typically comprised between 0.60 and 0.80 g/ml, preferably between 0.62 and 0.70 g/ml.

Furthermore, said coarse particles could be characterized for convexity. Convexity is a measurement of the edge roughness of a particle. It is calculated by dividing the convex hull perimeter by the actual particle perimeter. The easiest way to visualize the convex hull perimeter is to imagine an elastic band placed around the particle. Convexity also has values in the range 0-1. A smooth shape has a convexity of 1 while a very ‘spiky’ or irregular object has a convexity closer to 0. The shapes above show how convexity is unaffected by overall form; a smooth needle has the same convexity as a smooth circle. It can be determined as reported in Example 3. The convexity for the aforementioned particles is typically comprised between 0.70 and 0.99, preferably between 0.80 and 0.98, more preferably between 0.90 and 0.96.

Advantageously, the ratio between the fraction of fine excipient particles a) and the fraction of coarse excipient particles b) could be comprised between 1:99 and 30:70 by weight, preferably between 10:90 and 20:80 by weight.

Advantageously, the fraction of fine excipient particles a) consists of particles of a physiologically acceptable excipient and particles of a suitable additive, wherein at least 90% of all the particles have a volume diameter lower than 15 micron, preferably lower than 12 micron.

The ratio between the excipient particles and the additive particles within the fraction a) may vary depending on the doses of the active ingredients.

The additive material may include a combination of one or more materials, and it may be selected from amino acids such as leucine and isoleucine or surface active substances such as stearate salts.

In a preferred embodiment, the additive is magnesium stearate as, due to its hydrophobicity, it is capable of improving the moisture resistance of dry powder formulations for inhalation as disclosed in WO 00/28979.

Advantageously, when magnesium stearate is used, the fraction of fine excipient particles a) is composed of 90 to 98% by weight of the excipient and 2 to 10% by weight of magnesium stearate. In a particular embodiment, the amount may be 98% of the excipient particles and 2% of magnesium stearate, by weight.

To further improve the resistance to humidity, it is preferred subjecting the mixture of the physiologically acceptable excipient with magnesium stearate to co-micronization in a mill as disclosed in WO 01/78693 rather than simply mixing them.

As it can be appreciated from Example 2, in comparison to a fraction of particles a) obtained by simple mixing, a powder formulation comprising said fine fraction obtained by co-micronization shows a significantly improved stability of the fine particle fraction (FPF) of the active ingredients on storage at 30° C. and 75% relative humidity.

Typically, the particles are co-micronized starting from excipient particles having a mass diameter lesser than 250 micron and magnesium stearate particles having a mass diameter lesser than 35 micron using a jet mill, preferably in inert atmosphere, for example under nitrogen.

As an example, alpha-lactose monohydrate commercially available such as Meggle D 30 or Spherolac 100 (Meggle, Wasserburg, Germany) could be used as starting excipient.

Optionally, the fraction of fine excipient particles a) may be subjected to a conditioning step according to the conditions disclosed in the pending application n. WO 2011/131663.

Although the time of treatment will generally depend on the starting particle size of the excipient particles and the desired size reduction to be obtained, it is preferably performed for at least one hour, preferably for at least two hours, even more preferably for four hours or more.

It has indeed been found that, the greater is the intimate contact of the magnesium particles with the fine excipient particles, the higher is the capability of magnesium stearate to protect the formulation from the detrimental effect of humidity.

Therefore, by performing co-micronization for a rather long time, it may be possible to reduce the amount of magnesium stearate to be used in the formulation, while maintaining a good resistance to humidity.

To determine the intimacy between the excipient particles and magnesium stearate in fine fraction a), the extent of coating could be determined.

Advantageously, magnesium stearate coats the surface of the excipient particles of fine fraction a) in such a way that the extent of the surface coating is higher than 20%, preferably higher than 50%, more preferably higher than 60%.

The extent to which the magnesium stearate coats the surface of the excipient particles may be determined by X-ray photoelectron spectroscopy (XPS), a well known tool for determining the extent as well as the uniformity of distribution of certain elements on the surface of other substances. In the XPS instrument, photons of a specific energy are used to excite the electronic states of atoms below the surface of the sample. Electrons ejected from the surface are energy filtered via a hemispherical analyser (HSA) before the intensity for a defined energy is recorded by a detector. Since core level electrons in solid-state atoms are quantized, the resulting energy spectra exhibit resonance peaks characteristic of the electronic structure for atoms at the sample surface.

Typically XPS measurements are taken on an Axis-Ultra instrument available from Kratos Analytical (Manchester, UK) using monochromated Al Kα radiation (1486.6 eV) operated at 15 mA emission current and 10 kV anode potential (150 W). A low energy electron flood gun is used to compensate for insulator charging. Survey scans, from which quantification of the detected elements are obtained, are acquired with analyser pass energy of 160 eV and a 1 eV step size. High-resolution scans of the C 1s, O 1s, Mg 2s, N 1s and Cl 2p regions are acquired with pass energy of 40 eV and a 0.1 eV step size. The area examined is approximately 700 μm×300 μm for the survey scans and a 110 μm diameter spot for the high-resolution scans.

In the context of the invention, by XPS, it is possible to calculate both the extent of coating and the depth of the magnesium sterate film around the lactose particles. The extent of magnesium stearate (MgSt) coating is estimated using the following equation:

$$\% MgSt coating = ( \% {Mg}_{sample} / \% {Mg}_{ref} ) \times 100$$

where
Mgₛₐₘₚₗₑ is the amount of Mg in the analysed mixture;
Mg_{ref} is the amount of Mg in the reference sample of commercially available MgSt.

Usually the values are calculated as a mean of two different measurements. Typically, an accuracy of 10% is quoted for routinely performed XPS experiments.

Alternatively, when the excipient particles are made of lactose, preferably of alpha-lactose monohydrate, the extent of surface coating may be determined by water contact angle measurement, and then by applying the equation known in the literature as Cassie and Baxter, for example cited at page 338 of Colombo I et al Il Farmaco 1984, 39 (10), 328-341 and reported below.

$$cos {ϑ}_{mixture} = {f}_{MgSt} cos {ϑ}_{MgSt} + {f}_{lactose} cos {ϑ}_{lactose}$$

where f_{MgSt} and f_{lactore} are the surface area fractions of magnesium stearate and of lactose;
ϑ_{MgSt} is the water contact angle of magnesium stearate;
ϑ_{lactose} is the water contact angle of lactose
ϑₘᵢₓₜᵤᵣₑ are the experimental contact angle values.

For the purpose of the invention, the contact angle may be determined with methods that are essentially based on a goniometric measurement. These imply the direct observation of the angle formed between the solid substrate and the liquid under testing. It is therefore quite simple to carry out, being the only limitation related to possible bias stemming from intra-operator variability. It should be, however, underlined that this drawback can be overcome by adoption of a fully automated procedure, such as a computer assisted image analysis. A particularly useful approach is the sessile or static drop method which is typically carried out by depositing a liquid drop onto the surface of the powder in form of disc obtained by compaction (compressed powder disc method).

Within the limits of the experimental error, a good consistency has been found between the values of extent of coating as determined by XPS measurements, and those as estimated by the theoretical calculations based on the Cassie and Baxter equation.

The extent to which the magnesium stearate coats the surface of the excipient particles may also be determined by scanning electron microscopy (SEM), a well-known versatile analytical technique.

Such microscopy may be equipped with an EDX analyzer (an Electron Dispersive X-ray analyzer), that can produce an image selective to certain types of atoms, for example magnesium atoms. In this manner it is possible to obtain a clear data set on the distribution of magnesium stearate on the surface of the excipient particles.

SEM may alternatively be combined with IR or Raman spectroscopy for determining the extent of coating, according to known procedures.

Since it is well known in the art that magnesium stearate, together with improving moisture resistance, is able of improving the aerosol performances, it may occur that the reduction of its amount in the formulation reduces the respirable fraction of the active ingredients.

To compensate for that, fissured coarse particles having a very narrow interval of tapped density values shall be used, i.e. comprised between 0.65 and 0.75 g/cm³.

In fact, as demonstrated in Table 4 of Example 4, it has indeed been found that by strictly controlling the tapped density of the fissured coarse particles, better aerosol performances could be obtained.

Other parameters such as the convexity factor and the dynamic density could also be used to select the coarse lactose particles with better performances.

Typically, the convexity of said selected lactose particles are comprised between 0.94 and 0.96 and the dynamic density between 0.64 and 0.67 g/ml.

The tapped density could be determined according to methods known in the art.

Typically, the tapped density is obtained by mechanically tapping a graduated measuring cylinder or vessel containing the powder sample according to European Pharmacopeia Ed. 7.0, 2.9.34.

Otherwise it might be measured as follows: a measuring cylinder is weighed on a top pan balance (2 place). Approximately 50 g powder is introduced into the measuring cylinder, and the weight is recorded. The measuring cylinder containing the powder is attached to a jolting volumeter (Jel Stampfvolumeter). The jolting volumeter is set to tap 200 times. During each tap, the measuring cylinder is raised and allowed to fall a set distance. After the 200 taps, the volume of the powder is measured. The tapping is repeated and the new volume measured. The tapping is continued until the powder will settle no more. The tapped density is calculated as the weight of the powder divided by the final tap volume. The procedure is performed three times (with new powder each time) for each powder measured, and the mean tapped density calculated from those three final tapped volume values.

Accordingly, in a preferred embodiment, the pharmaceutical composition filled in the multidose inhaler device comprises a fraction of fine excipients particles a) consisting of alpha-lactose monohydrate and magnesium stearate in amounts comprised between 98 and 99.0% and between 2.0 and 1.0% by weight, respectively; a fraction of coarse excipient particles b) consisting of alpha-lactose monohydrate having a particle size comprised between 210 and 360 micron and a tapped density comprised between 0.65 and 0.75 g/cm³, being the ratio between the fraction of fine excipient particles a) and the fraction of coarse excipient particles b) comprised between 2.5:97.5 and 7.5:92.5 by weight, and micronized particles of a pharmaceutically acceptable salt of formoterol, in combination with a pharmaceutically acceptable salt of glycopyrronium, wherein the fraction of fine particles a) is obtained by co-micronization by mixing for at least 1 hour, preferably for at least 2 hours.

In a particular embodiment, co-micronization is performed by mixing for a time comprised between 2 and 4 hours. It turns out that the percentage of magnesium stearate in the formulation would be comprised between 0.02 and 0.1 percent by weight.

The mixing of the fraction of coarse particles b) with the fraction of fine particles a) is typically carried out in suitable mixers, e.g. tumbler mixers such as Turbula™ or Dynamix™, rotary mixers, or instant mixer such as Diosna™, for at least 5 minutes, preferably for at least 30 minutes, more preferably for at least 2 hours.

In a general way, the person skilled in the art shall adjust the time of mixing and the speed of rotation of the mixer to obtain a homogenous mixture.

When spheronized coarse excipient particles are desired to obtain hard-pellets according to the definition reported above, the step of mixing shall be typically carried out for at least four hours.

The active ingredients shall be present in micronized form.

Advantageously, at least 90% of all said micronized particles of the active ingredients have a volume diameter lower than 6.0 micron, preferably comprised between than 5.5 and 4.0 micron, and the volume median diameter of said particles is comprised between 1.2 and 2.5 micron, preferably between 1.3 and 2.2 micron. More advantageously, no more than 10% of all said micronized particles of the active ingredients have a diameter lower than 0.6 micron, preferably equal to or lower than 0.7 micron, more preferably equal to or lower than 0.8 micron. In a particular embodiment, no more than 10% of all said micronized particles of the active ingredients have a diameter comprised between 0.6 and 1.0 micron.

From the above particle size distribution, it follows that the width of the particle size distribution of the particles of each active ingredient, expressed as a span, should be advantageously comprised between 1.0 and 4.5, more advantageously between 1.2 and 3.0, preferably between 1.3 and 2.1, more preferably between 1.6 and 2.0. According to Chew et al J Pharm Pharmaceut Sci 2002, 5, 162-168, the span corresponds to [d(v, 0.9)−d(v,0.1)]/d (v,0.5).

Even more advantageously, at least 99% of said particles [d(v,0.99)] shall have a volume diameter equal to or lower than 7.0 micron, and substantially all the particles have a volume diameter comprised between 6.8 and 0.4 micron, preferably between 6.5 and 0.45 micron.

The size of the active particles could be determined by measuring the characteristic equivalent sphere diameter, known as volume diameter, by laser diffraction. In the reported examples, the volume diameter has been determined according to European Pharmacopeia Ed. 7.0, 2.9.31, pp 295-298, using a Malvern apparatus under wet conditions, i.e. by suspending the particles in water in the presence of a surfactant. By referring to the manual of the apparatus, the skilled person in the art shall have all the information about how to operate.

The step of mixing the fraction of the carrier particles with all the micronized active ingredients may be carried out according to methods known in the art, for example by mixing the components in suitable known apparatus, such as a Turbula™ or Dynamix™ mixer for a sufficient period to achieve the homogeneity of the active ingredient in the final mixture.

Typically, the mixing is carried out for a time comprised between 30 and 120 minutes, preferably between 45 and 100 minutes.

The pharmaceutical composition of the invention may be suitable for delivering a therapeutic amount of all active ingredients in one or more actuations (shots or puffs) of the inhaler.

For example, the formulations will be suitable for delivering 3-12 microg formoterol (as fumarate dihydrate) per actuation, especially 6 microg or 12 microg per actuation, and when present, 6-50 microg glycopyrronium (as bromide), especially 12.5 or 25 microg, and 25-200 microg beclometasone dipropionate (BDP) per actuation, especially 50, 100 or 200 microg per actuation.

Accordingly, the concentration of formoterol (as fumarate dihydrate) in the formulation could be comprised between 0.03 and 0.12% by weight, and when present, those of glycopyrronium (as bromide) and BDP could be comprised between 0.06 and 0.50 and 0.35 and 2.0% percent by weight, respectively.

Administration of the formulations of the invention is preferably indicated for the prevention and/or treatment of chronic obstructive pulmonary disease (COPD). However, said formulation might also be indicated for the prevention and/or treatment of asthma of all types and severity, including severe persistent asthma, as well as further respiratory disorders characterized by obstruction of the peripheral airways as a result of inflammation and presence of mucus such as chronic obstructive bronchiolitis.

In certain embodiments, the formulations of the invention are suitable for the prevention and/or treatment of severe and/or very severe forms of respiratory disorders, in particular severe and/or very severe forms of COPD.

The term “drug product” is to be construed to encompass the multidose dry powder inhalation device, filled with the pharmaceutical composition above described in the reservoir chamber, and molecular sieves in the desiccant chamber, and optionally a pouch which encloses the dry powder inhalation device.

As it can be appreciated from FIG. 4, the pouch contributes to reduce the absorption of moisture.

In a preferred embodiment, the pouch is a low-moisture permeable package, for example the one disclosed in EP 1760008.

The invention is illustrated in detail by the following examples.

### EXAMPLES

### Example 1—Effect of the Molecular Sieves

Eleven DPI devices as those disclosed in WO 2016/000983 were filled with 0.5 g of molecular sieves (Propagroup, Italy) in the desiccant chamber and stored at 30° C. and 75% relative humidity for 21 days (PROPAGROUP). For comparison, five DPI devices filled with silica gel in the desiccant chamber were stored under the same conditions (STD).

The weight increment was determined weekly as considered representative of the absorbed humidity in the unit of time, and hence of the tendency of the device to remain more dry. The results are plotted in FIG. 3 and show that molecular sieves absorb humidity much quicker, allowing for a more dry environment to be maintained.

### Example 2—Simple Mixing Vs Co-Micronization: Effect on Stability

About 775 g particles of alpha-lactose monohydrate having a particle size of less than 250 micron (Meggle D 30, Meggle), and 2% magnesium stearate particles (Peter Greven, Germany) were co-micronized (ratio 98:2 by weight) by milling in a jet mill operating under nitrogen for 4 hours to obtain the fraction of fine particles a).

The mixing was carried out in a Dynamix mixer operating at a rotation speed of 16 r.p.m. around two different rotating axis for a period of 240 minutes.

The resulting mixtures of particles are termed hereinafter as Carrier.

The Carrier as obtained above was mixed with micronized formoterol fumarate dihydrate (FF) and glycopyrronium bromide (GB) in a Dynamix mixer for 54 minutes at 18 r.p.m. and at 10 r.p.m. around two different rotating axis.

Micronized beclometasone dipropionate (BDP) was further added and blended in a Dynamix mixer for 72 minutes at 24 r.p.m. and at 16 r.p.m. around two different rotating axis to obtain the final formulation.

The final formulation was passed through a sieve with mesh size 0.6 mm (600 micron) and finally blended in a Dynamix mixer for 21 minutes at 16 r.p.m.

The ratio of the active ingredients to 10 mg of the carrier is 6 microg of FF dihydrate, 100 microg of BDP and 12.5 microg of glycopyrronium bromide.

For comparison, a powder formulation wherein the Carrier is obtained by simply mixing the components was obtained as reported in Examples 1 and 2 of WO 2017/085004.

The powder formulations were stored at 30° C. and 75% relative humidity. The aerosol performances were determined after loading it in the multidose dry powder inhaler described in WO 2016/000983.

The evaluation of the aerosol performance was carried out using the Next Generation Impactor (NGI) according to the conditions reported in the European Pharmacopeia 6^{th} Ed 2008, par 2.9.18, pages 293-295.

The following parameters, were calculated: i) the delivered dose which is the amount of drug delivered from the device recovered in all the parts of impactor; ii) the fine particle mass (FPM) which is the amount of delivered dose having a particle size equal to or lower than 5.0 micron and the extrafine FPM which is the amount of delivered dose having a particle size equal to or lower than 2.0 micron; iii) the fine particle fraction (FPF) which is the percentage of the fine particle dose.

The results as a mean of the FPM are reported in Table 1.

TABLE 1

| Stability check point | Mean values - simple mixing process | | |
|---|---|---|---|
| in use at 30° C./75% | BDP | FF | GB |
| (months) | FPM drop (%) | FPM drop (%) | FPM drop (%) |
| T = 0 M | — | — | — |
| T = 0 + 1 M* | −22.1 | −20.7 | −24.5 |
| T = 0 + 1.5 M | −30.5 | −30.0 | −36.5 |
| T = 0 + 2 M | −26.3 | −23.1 | −29.5 |
| T = 0 + 3 M | −30.2 | −26.6 | −33.1 |
| T = 0 M | — | — | — |
| T = 0 + 1 M | −10.4 | −7.8 | −7.8 |
| T = 0 + 2 M | −20.3 | −19.1 | −19.1 |
| T = 0 + 3 M | −19.6 | −20.7 | −20.7 |

As it can be appreciated, a powder formulation comprising the fine fraction of particles a) obtained by co-micronization shows a minor drop and hence a significantly improved stability of the fine particle mass (FPM) for all the three active ingredients upon storage at 30° C. and 75% relative humidity.

### Example 3—Technological Characterization of the Coarse Lactose Particles

Different samples of fissured coarse particles of alpha-lactose monohydrate having a mass diameter comprised between 212-355 micron were characterized for bulked and tapped density, dynamic density and convexity.

The bulked and tapped densities were determined according to European Pharmacopeia Ed. 7.0, 2.9.34 using a 250 ml cylinder.

The dynamic density was assessed using a 16 ml cell and a powder flowability tester (Mercury Scientific Inc, Italy) according to the following parameters; avalanche count: 99, rotation rate: 0.3 rpm, preparation time: 60 sec. imag. rate: 10 fp; data points: 5299, avalanche threshold: 0.65%; angle calculation: half, shutter speed: 7 ms, image threshold: 150; gain: 8.00.

The convexity was determined by using a Morphologi G3 equipment (Malvern Instruments Inc, UK) and according to the following parameters: sample amount: 38 mm³; pressure of dispersion: 0.5 bar; injection time: 20 ms; settling time: 180 s; total scanned area: circle with 4.2 cm radius; optics: 2.5×.

Only particles above 200 μm were measured and included in the shape evaluation; if two or more particles were found to be close to each other in a single image, this was excluded from the equipment evaluation.

The results expressed as mean and RSD are reported in Tables 2 and 3.

TABLE 2

| | Dynamic | Bulk | Tapped | |
|---|---|---|---|---|
| | density | density | density | |
| Batch A | (g/mL) | (g/mL) | (g/mL) | Convexity |
| 1 | 0.679 | 0.66 | 0.71 | 0.942 |
| 2 | 0.670 | 0.66 | 0.72 | 0.944 |
| 3 | 0.662 | — | — | 0.950 |
| 4 | 0.667 | 0.64 | 0.71 | 0.937 |
| 5 | 0.654 | 0.65 | 0.72 | 0.939 |
| 6 | 0.662 | — | — | 0.948 |
| 7 | 0.663 | 0.64 | 0.71 | — |
| 8 | 0.658 | 0.65 | 0.72 | — |
| 9 | — | — | — | — |
| 10 | 0.642 | 0.64 | 0.71 | — |
| 11 | 0.643 | 0.64 | 0.72 | — |
| Mean | 0.660 | 0.65 | 0.72 | 0.943 |
| RSD | 0.011 | 0.01 | 0.01 | 0.005 |

TABLE 3

| | Dynamic | Bulk | Tapped | |
|---|---|---|---|---|
| | density | density | density | |
| Batch B | (g/mL) | (g/mL) | (g/mL) | Convexity |
| 1 | 0.615 | 0.60 | 0.68 | 0.932 |
| 2 | 0.616 | 0.60 | 0.68 | 0.925 |
| 3 | 0.616 | — | — | 0.923 |
| 4 | 0.616 | 0.60 | 0.69 | 0.928 |
| 5 | 0.616 | 0.61 | 0.69 | 0.926 |
| 6 | 0.616 | — | — | 0.923 |
| 7 | 0.609 | 0.60 | 0.68 | — |
| 8 | 0.609 | 0.60 | 0.69 | — |
| 9 | 0.608 | — | — | — |
| 10 | 0.602 | 0.60 | 0.69 | 0.930 |
| 11 | 0.601 | 0.60 | 0.69 | 0.921 |
| 12 | 0.607 | — | — | 0.915 |
| Mean | 0.611 | 0.60 | 0.69 | 0.925 |
| RSD | 0.005 | 0.00 | 0.00 | 0.005 |

As it can be appreciated, although the differences are small, the values are significantly different, in particular for the tapped density.

### Example 4—Effect of the Tapped Density

Samples were prepared as reported in Example 2 using a fraction of fine particles a) obtained by co-micronization and representative samples of batches A and B of coarse lactose particles.

The evaluation of the aerosol performance was carried out as reported in Example 2.

The results are reported in Table 4.

TABLE 4

| Sample | B | A |
|---|---|---|
| Dynamic | 0.611 +/− 0.005 | 0.660 +/− 0.011 |
| density | | |
| (g/mL) | | |
| Bulk density | 0.60 +/− 0.00 | 0.65 +/− 0.01 |
| (g/mL) | | |
| Tapped | 0.69 +/− 0.00 | 0.72 +/− 0.01 |
| density | | |
| (g/mL) | | |
| Convexity | 0.925 +/− 0.005 | 0.943 +/− 0.005 |

| | Value | Mean | SD | Mean | SD |
|---|---|---|---|---|---|
| | SW1 | 9.9 | 0.6 | 10.3 | 0.3 |
| FF | FPM | 2.0 | 0.1 | 2.7 | 0.2 |
| | FPF | 36.8 | 2.1 | 48.4 | 2.5 |
| | DD | 5.4 | 0.3 | 5.6 | 0.1 |
| | MDD | 5.4 | 0.1 | 5.6 | 0.1 |
| GB | FPM | 3.4 | 0.2 | 4.8 | 0.3 |
| | FPF | 30.9 | 1.7 | 41.3 | 2.4 |
| | DD | 11.0 | 0.5 | 11.4 | 0.1 |
| | MDD | 11.1 | 0.2 | 11.7 | 0.1 |
| BDP | FPM | 29.5 | 1.3 | 41.6 | 2.6 |
| | FPF | 33.4 | 1.9 | 45.0 | 2.2 |
| | DD | 88.6 | 4.4 | 90.4 | 6.5 |
| | MDD | 88.5 | 1.3 | 92.4 | 0.6 |

The results demonstrate that powder formulation comprising coarse lactose particles having a higher tapped density give rise to better aerosol performance for all three active ingredients.

### Example 5—Effect of the Improvement of the Invention on the Absorption of Humidity

DPI devices as those disclosed in WO 2016/000983, separately incorporating the modifications of the invention were stored at 35° C. and 75% relative humidity for at least 14 days. The weight increment was determined weekly as considered representative of the absorbed humidity in the unit of time, and hence of the tendency of the device to remain more dry. The results are plotted in FIG. 4, which shows a differential weight over time due to moisture absorption of inhalers incorporating different separate embodiments of the invention compared to a standard inhaler. As it can be appreciated, all the improvements of the invention are effective in decreasing the absorption of moisture.

1.-17. (canceled)

18. A drug product comprising a multidose dry powder inhalation device having a pharmaceutical composition within,
the dry powder inhalation device comprising a medicament chamber, and a desiccant chamber adjacent to the medicament chamber and filled with molecular sieves, and
the pharmaceutical composition comprising a pharmaceutically acceptable salt of formoterol as active ingredient, together with an excipient.

19. The drug product according to claim 18, wherein the pharmaceutical composition further comprises a pharmaceutically acceptable salt of glycopyrronium and/or a pharmaceutically acceptable salt of beclometasone dipropionate as active ingredients.

20. The drug product according to claim 19, wherein the pharmaceutically acceptable salt of glycopyrronium is a bromide salt of glycopyrronium.

21. The drug product according to claim 18, wherein the molecular sieves are made of alkaline salts of aluminosilicates, with pores having a diameter from 2 to 50 Angstrom.

22. The drug product according to claim 21, wherein the pores have a diameter from 3 to 20 Angstrom.

23. The drug product according to claim 18, wherein the multidose dry powder inhalation device comprises:
a casing having a mouthpiece and delimiting an inhalation channel connected to an opening of the mouthpiece;
a container for storing a powdered medicament, the container being placed in the casing;
a dispensing device placed in the casing and configured to dispense unit doses of the powdered medicament from the container to the inhalation channel for inhalation through the mouthpiece; and
a cover engageable with the casing to close the mouthpiece, the cover comprising a sealing element for humidity resistance,
wherein, when the cover is engaged with the casing and closes the mouthpiece, a main portion of the sealing element is coupled to the opening to tight close said opening.

24. The drug product according to claim 23, wherein the sealing element comprises a material that is more deformable than a material of the mouthpiece.

25. The drug product according to claim 23, wherein the sealing element has a hardness between 10 Shore A and 60 Shore A, between 20 Shore A and 50 Shore A, or between 25 Shore A and 35 Shore A.

26. The drug product according to claim 23, wherein the sealing element comprises or is made of silicone.

27. The drug product according to claim 23, wherein the sealing element is over-molded to the cover or push-fitted in the cover.

28. The drug product according to claim 27, wherein the sealing element is glued to the cover.

29. The drug product according to claim 18, wherein the pharmaceutical composition comprises the active ingredient in micronized form.

30. The drug product according to claim 18, wherein the excipient comprises
a fraction of fine excipient particles consisting of a physiologically acceptable excipient and magnesium stearate;
a fraction of coarse excipient particles made of a physiologically acceptable excipient and having a particle size comprised between 200 and 400 micron,
wherein the fraction of fine excipient particles is obtained by micronization upon mixing for at least 1 hour.

31. The drug product according to claim 30, wherein the physiologically acceptable excipient is alpha-lactose monohydrate.

32. The drug product according to claim 18, wherein the excipient comprises
a fraction of fine excipient particles consisting of alpha-lactose monohydrate and magnesium stearate in amounts comprised between 98 and 99.0% and between 2.0 and 1.0% by weight, respectively; and
a fraction of coarse excipient particles consisting of alpha-lactose monohydrate having a particle size comprised between 210 and 360 micron and a tapped density comprised between 0.65 and 0.75 g/cm³,
wherein a ratio between the fraction of fine excipient particles and the fraction of coarse excipient particles is comprised between 2.5:97.5 and 7.5:92.5 by weight.

33. The drug product according to claim 18, further comprising a pouch enclosing the dry powder inhalation device.

34. A process for manufacturing the drug product according to claim 18, said process comprising:
filling the medicament chamber of a multidose dry powder inhalation device with the pharmaceutical composition comprising a pharmaceutically acceptable salt of formoterol, optionally in combination with a pharmaceutically acceptable salt of glycopyrronium and/or beclometasone dipropionate, and
filling the desiccant chamber of said dry powder inhalation device with molecular sieves.
